(19) United States
(12) Patent Application Publication
(10) Pub. No.: US 2008/0051864 A1
(43) Pub. Date: Feb. 28, 2008

(54) EPICARDIAL LEAD

(75) Inventors: Peter L. CALLAS, Castro Valley, CA (US); John S. GREENLAND, San Diego, CA (US); Ronald W. HEIL, Roseville, MN (US); Randy W. WESTLUND, River Falls, WI (US); Peter T. KELLEY, Buffalo, MN (US)

Correspondence Address: FAEGRE & BENSON, LLP;BOSTON SCIENTIFIC PATENT DOCK, 2200 WELLS FARGO CENTER, 90 SOUTH SEVENTH STREET, MINNEAPOLIS, MN, 55402-3901, US

(73) Assignee: CARDIAC PACEMAKERS, INC., St. Paul, MN (US)

(21) Appl. No.: 11/466,271

(22) Filed: Aug. 22, 2006

(51) Int. Cl. A61N 1/05

(52) U.S. Cl. 607/130

(57) ABSTRACT

A lead for implanting into the epicardium includes a pair of tissue anchors coupled to a tissue engaging member, forming an anchor mechanism. The tissue anchors include electrodes coupled to conductors extending from the tissue engaging member. The tissue anchors are movable from a low profile configuration to an implanting configuration in which the tissue anchors are angled away from the tissue engaging member. A device for implanting the lead includes one or more lumens, including a lead lumen and a vacuum lumen terminating at a distal opening in the device. Suction is applied at the distal opening through the vacuum lumen to draw an epicardial bleb. The anchor mechanism of the lead is withdrawn proximally past the bleb, causing the tissue anchors to pierce the epicardium. The device is then withdrawn proximally over the conductors.

### TECHNICAL FIELD

This invention relates generally to implantable lead assemblies for stimulating and/or sensing electrical signals in muscle tissue. More particularly, it relates to myocardially-implanted leads for cardiac stimulation and systems for inserting and anchoring the leads.

### BACKGROUND

Cardiac rhythm management systems are used to treat heart arrhythmias. Pacemaker systems, for example, are commonly implanted in patients to treat bradycardia (i.e., abnormally slow heart rate). A pacemaker system includes an implantable pulse generator and leads, which form the electrical connection between the implantable pulse generator and the cardiac muscle of the heart. Another example are implantable cardioverter defibrillator (“ICD”) systems, used to treat tachycardia (i.e., abnormally rapid heart rate). An ICD system also includes a pulse generator and leads that deliver electrical energy to the heart.

The leads coupling the pulse generator to the cardiac muscle are commonly used for delivering an electrical pulse to the cardiac muscle, for sensing electrical signals produced in the cardiac muscle, or for both delivering and sensing. The leads are susceptible to categorization according to the type of connection they form with the heart. An endocardial lead includes at least one electrode at or near its distal tip adapted to contact the endocardium (i.e., the tissue lining the inside of the heart). An epicardial lead includes at least one electrode at or near its distal tip adapted to contact the epicardium (i.e., the tissue lining the outside of the heart). Finally, a myocardial lead includes at least one electrode at or near its distal tip inserted into the heart muscle or myocardium (i.e., the muscle sandwiched between the endocardium and epicardium). Some leads have multiple spaced apart distal electrodes at differing polarities and are known as bipolar type leads. The spacing between the electrodes can affect lead performance and the quality of the electrical signal transmitted or sensed through the heart tissue.

The lead typically consists of a flexible conductor surrounded by an insulating tube or sheath that extends from the electrode at the distal end to a connector pin at the proximal end. Endocardial leads are typically delivered transvenously to the right atrium or ventricle and commonly employ tines at a distal end for engaging the trabeculae.

The treatment of congestive heart failure, however, often requires left ventricular stimulation either alone or in conjunction with right ventricular stimulation. For example, cardiac resynchronization therapy (also commonly referred to as biventricular pacing), an emerging treatment for heart failure, requires stimulation of both the right and the left ventricle to increase cardiac output. Left ventricular stimulation requires placement of a lead in or on the left ventricle near the apex of the heart. One technique for left ventricular lead placement is to expose the heart by way of a thoracotomy. The lead is then positioned so that one or more electrodes contact the epicardium or are embedded in the myocardium. Another method is to advance an epicardial lead endovenously into the coronary sinus and then advance the lead through a lateral vein of the left ventricle. The electrodes are positioned to contact the epicardial surface of the left ventricle.

The left ventricle beats forcefully as it pumps oxygenated blood throughout the body. Repetitive beating of the heart, in combination with patient movement, can sometimes dislodge the lead from its implanted position in the cardiac muscle. The electrodes may lose contact with the cardiac muscle, or the spacing between electrodes may alter over time.

There is a need for an improved pacing lead suitable for chronic implantation and a minimally invasive delivery system and method for implanting such a lead.

### SUMMARY

In one embodiment, the present invention is an epicardial lead including an insulated conductor having a proximal end and a distal end, an anchor assembly coupled to the distal end of the conductor and an electrode positioned on the anchor assembly and in electrical communication with the conductor. The anchor assembly includes a tissue engaging member and a tissue anchor having a first end coupled to the tissue engaging member and a second end movable relative to the tissue engaging member. The second end of the tissue anchor is biased away from the tissue engaging member to a position spaced apart from the tissue engaging member.

In another embodiment, the present invention is a cardiac rhythm management system including a pulse generator for delivering therapy to a patient's heart, an insulated conductor, an anchor assembly and an electrode. The conductor has a proximal end coupled to the pulse generator and a distal end adapted for implantation in the patient's heart. The anchor assembly is coupled to the distal end of the conductor, and includes an anchor means coupled to a tissue engaging member. The electrode is positioned on the anchor assembly and is in electrical communication with the conductor.

In yet another embodiment, the present invention is a method of implanting a lead into a space between a pericardium and an epicardium of a heart with a delivery device. A distal end of the delivery device is advanced into the space between the pericardium and the epicardium. The lead is withdrawn proximally relative to the delivery device such that a tissue anchor on a distal end of the lead is biased away from the lead into engagement with the epicardium. The lead is tensioned such that the tissue anchor penetrates the myocardium and the epicardium is wedged between the tissue anchor and a distal end of the lead.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a lead according to one embodiment of the invention, in relation to a heart.

FIG. 2 is a perspective view of a distal end portion of a lead according to one embodiment of the invention.

FIG. 3A is a side view of the lead of FIG. 2 in which the tissue anchors are in a compressed position.

FIG. 3B is a side view of the lead of FIG. 2 in which the tissue anchors are biased outwardly.

FIG. 4A is a front view of the anchor mechanism of FIG. 2 in which the tissue anchors are in a compressed position.

FIG. 4B is a front view of the anchor mechanism of FIG. 2 in which the tissue anchors are biased outwardly.

FIG. 4C is an angled view of the underside of the anchor mechanism of FIG. 4B.

FIG. 5A is a perspective view of an anchor mechanism according to another embodiment of the invention.

FIG. 5B is a front view of the anchor mechanism of FIG. 5A.

FIG. 6 is a perspective view of an anchor mechanism according to another embodiment of the present invention.

FIG. 7 is a perspective view of an anchor mechanism according to another embodiment of the present invention.

FIG. 8 is a perspective view of an anchor mechanism according to another embodiment of the present invention.

FIG. 9 is a side view of a delivery device for use in delivering a lead according to various embodiments of the present invention.

FIG. 10A is a perspective view of a distal portion of the delivery device of FIG. 9.

FIG. 10B is a sectional view of the delivery device of FIG. 10B taken along line 10-10.

FIG. 11 is a flowchart illustrating the steps for a method of inserting an epicardial lead into the heart according to one embodiment of the present invention.

FIG. 12 is a side view of an assembled lead and delivery device shown in relation to the anatomic layers of the heart.

FIG. 13 shows a myocardial bleb drawn into the delivery device of FIG. 12.

FIG. 14 shows the epicardial lead partially inserted into the myocardial bleb of FIG. 13.

FIG. 15 shows the delivery device being withdrawn over the epicardial lead of FIG. 14.

FIG. 16 is a perspective view of a distal portion of a delivery device for use in delivering a lead according to another embodiment of the invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

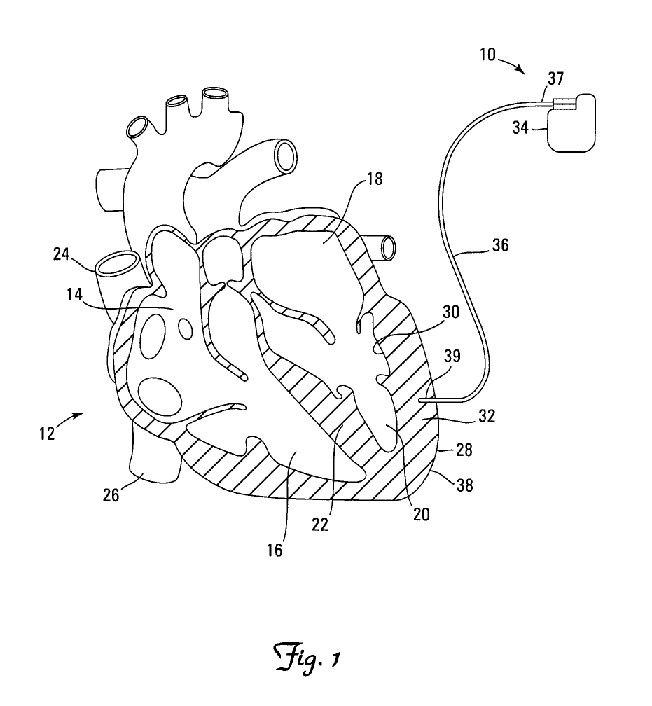

FIG. 1 shows a cardiac rhythm management system 10 deployed in a human heart 12 according to one embodiment of the present invention. The heart 12 includes a right atrium 14 and a right ventricle 16 separated from a left atrium 18 and a left ventricle 20 by a septum 22. During normal operation of the heart 12, deoxygenated blood is fed into the right atrium 14 through the superior vena cava 24 and the inferior vena cava 26. The deoxygenated blood flows from the right atrium 14 into the right ventricle 16. The deoxygenated blood is pumped from the right ventricle 16 into the lungs, where the blood is re-oxygenated. From the lungs the oxygenated blood flows into the left atrium 18, then into the left ventricle 20. The left ventricle 20 beats forcefully to pump the oxygenated blood throughout the body.

The outer walls of the heart 12 are lined with a tissue known as the epicardium 28. The inner walls of the heart are lined with a tissue known as the endocardium 30. The heart muscle, or myocardium 32, is sandwiched between the endocardium 30 and the epicardium 28. A tough outer pericardial sac (not shown) surrounds the heart 12.

The cardiac rhythm management system 10 includes a pulse generator 34 coupled to an epicardial lead 36. The pulse generator 34 is typically implanted in a pocket formed underneath the skin of the patient's chest or abdominal region. The pulse generator 34 may be any of a variety of implantable devices known in the art for sensing electrical activity of the heart 12 and/or for delivering therapy to the heart 12. The lead 36 extends from a proximal end 37 couplable to the pulse generator 34 to a distal end 39 implanted in the myocardium 32 near an apex 38 of the heart 12. The lead 36 delivers electrical signals from the pulse generator 34 to an electrode located at or near the distal end 39 to accomplish pacing of the heart 12.

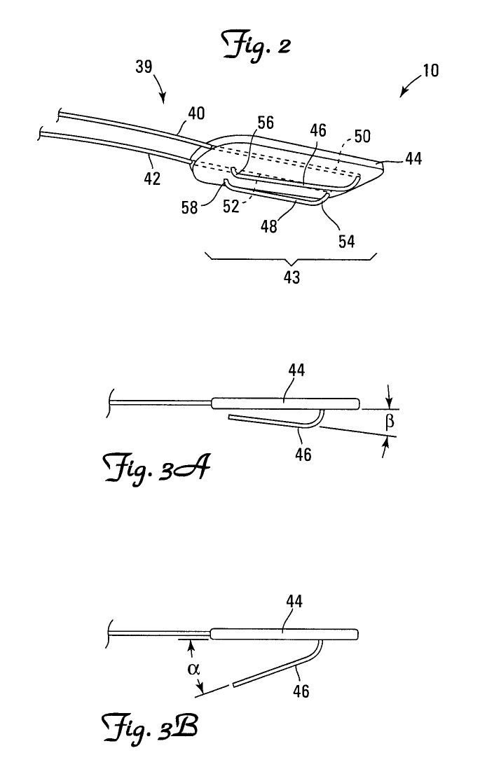

FIG. 2 shows the distal end 39 of the epicardial lead 36 in greater detail according to one embodiment of the present invention. The epicardial lead 36 includes a pair of insulated conductive members 40, 42 coupled to an anchor mechanism 43. The conductors 40, 42 each have a proximal end (not shown) which may be coupled to the pulse generator 34 and a distal end 50, 52 coupled to the anchor mechanism 43. The conductors 40, 42 may be insulated wires, cables or conductive coils and may be bundled with one another as shown in FIG. 1, or separate from one another, as shown in FIG. 2.

The anchor mechanism 43 operates to secure the lead 36 to the heart 12. As shown in FIG. 2, the anchor mechanism 43 includes a tissue engaging member 44, and a pair of tissue anchors 46, 48 coupled to the tissue engaging member 44. The tissue engaging member 44 acts as a brace against the heart 12 while the tissue anchors 46, 48 are inserted into the tissue of the heart.

As shown, the tissue engaging member 44 has a tissue engaging surface 51 facing the surface of the heart 12. The tissue engaging member 44 is plate-like and generally planar. In other embodiments, however, the tissue engaging member 44 has an arcuate cross-sectional shape. For example, the tissue engaging member 44 may have a curved profile complementary to the outer profile of the heart 12. Alternately, only the tissue engaging surface 51 may have a curved profile. The tissue engaging member 44 is shown in FIG. 2 as generally rectangular with curved corners. The tissue engaging member 44, however, may have any other shape. For example, the tissue engaging member 44 may be shaped like a square, circle, oval, or more complex shape.

The tissue anchors 46, 48 are pin-shaped members adapted for insertion into the heart 12 and for gripping tissue such as the myocardium 32. A first or distal end 53, 54 of the tissue anchors 46, 48 are coupled to the tissue engaging member 44. A second or proximal end 56, 58 of the tissue anchors 46, 48 are separate and movable relative to the tissue engaging member 44. Distal and proximal in this context are measured relative to the lead 36 overall.

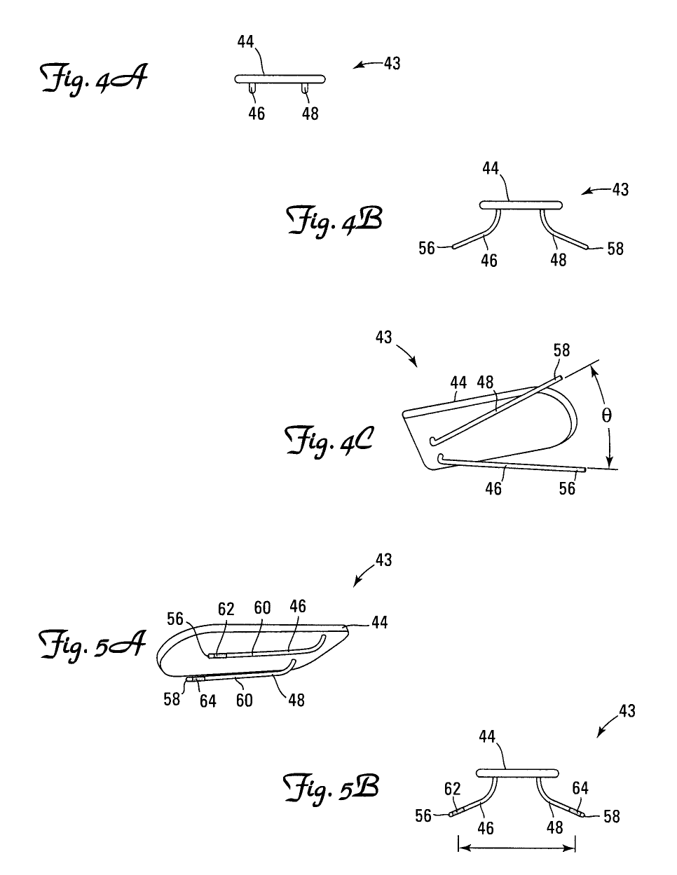

In the embodiment shown in FIG. 2, the proximal ends 56, 58 of the tissue anchors 46, 48 are movable relative to the tissue engaging member 44 in two directions. First, the proximal ends 56, 58 of the tissue anchors 46, 48 are movable away from the tissue engaging member 44 from a first position, as shown in FIG. 3A, to a second position, as shown in FIG. 3B. Second, the proximal ends 56, 58 of the tissue anchors 46, 48 are movable away from one another from the first position shown in FIG. 4A, to the second, spaced-apart position shown in FIG. 4B.

When the tissue anchors 46, 48 are in the compressed or first position, the distal end 39 of the lead 36 has a low profile adapted for insertion into the patient. The tissue anchors 46, 48 are positioned adjacent the tissue engaging member 44. In the embodiment generally illustrated in FIGS. 2 and 4A, the tissue anchors 46, 48 are approximately parallel to the tissue engaging member 44 in the first position. In other embodiments, however, the tissue anchors 46, 48 may be angled slightly towards or away from the tissue engaging member 44 when in the first position. In the embodiment generally illustrated in FIG. 3A, the tissue anchors 46, 48 are angled towards the tissue engaging member 44 at an angle β of about 2° when in the first position. In other embodiments, the angle β may be from about 5° towards the tissue engaging member 44 to about 5° away from the tissue engaging member 44. In addition, when in the first position, the tissue anchors 46, 48 may be generally parallel to one another, as illustrated in FIGS. 2 and 4A, or slightly towards or away from one another.

When the tissue anchors 46, 48 are in the expanded or second position, the tissue engaging member 44 and the tissue anchors 46, 48 are operable to be inserted into the heart 12 to secure the distal end 39 of the lead 36 to the heart 12. As discussed previously, the tissue engaging member 44 then acts as a brace, preventing proximally directed movement of the lead 36 away from its implanted position. In the embodiment illustrated in FIG. 3B, the tissue anchors 46, 48 are angled away from the tissue engaging member 44 at an angle α of about 35°. In other embodiments, however, the angle α may be from about 25° to about 50°. In the second position, as illustrated in FIG. 4C, the tissue anchors 46, 48 are angled away from one another at an angle θ of about 40°. In other embodiments, however, the angle θ may be from about 90°, 180° or anywhere generally between about 30° and 180°.

In general, increase the angle θ between the tissue anchors 46, 48 when in the second position increases the self-retention of the anchor mechanism into the tissue regardless of the angle α between the tissue anchors 46, 48. However, increasing the angle α between the tissue anchors 46, 48 increases the distance between the tissue anchors 46, 48, which may be used to control electrode spacing, as discussed with respect to the embodiment generally shown in FIGS. 5A and 5B.

In one embodiment, the distal ends 53, 54 of the tissue anchors 46, 48 are flexible. This flexibility permits the tissue anchors 46, 48 to move relative to the tissue engaging member 44 such that the proximal ends 56, 58 of the tissue anchors 46, 48 are positioned adjacent the tissue engaging member 44 or spaced apart from the tissue engaging member 44. In other embodiments, the tissue anchors 46, 48 may be pivotally or hingedly coupled to the tissue anchor 44.

In one embodiment, the tissue anchors 46, 48 are biased towards the second position, or outwardly or away from the tissue engaging member 44. This biasing causes the tissue anchors 46, 48 to tend to move away from the tissue engaging member 44 towards the second position in the absence of a force retaining them in proximity with the tissue engaging member 44.

In one embodiment, the tissue anchors 46, 48 are electrically coupled to the conductors 40, 42. In the embodiment shown in FIG. 2, the tissue anchors 46, 48 are exposed such that the entire tissue anchor 46, 48 forms an electrode. In another embodiment shown FIG. 5A, an insulated coating 60 covers the tissue anchors 46, 48 except for one or more exposed regions forming electrodes 62, 64. In the embodiment shown generally in FIG. 2, the electrodes 60, 62 are formed at the proximal ends 56, 58 of the tissue anchors 46, 48. The electrodes 62, 64, however, may be formed anywhere on the tissue anchors 46, 48. In addition, multiple electrodes may be formed on each tissue anchor (not shown).

In the spaced-apart position, in the embodiment shown in FIG. 5B, the horizontal distance between the proximal ends 56, 58 of the tissue anchors 46, 48 is about 1 cm. The electrodes 62, 64 are thus also spaced apart by about 1 cm. This spacing is thought to provide sufficient spacing for bipolar sensing and pacing of the myocardium 32. However, the horizontal spacing may be increased or decreased to provide greater or lesser distance between the electrodes 62, 64 as desired. In addition, the electrodes 62, 64 may be positioned more proximally or more distally on the tissue anchors 46, 48 to adjust the horizontal spacing between the electrodes 62, 64 and the depth of penetration of the electrodes 62, 64 into the myocardium 32. By adjusting the position of the electrodes 62, 64 on the tissue anchors 46, 48 and the spatial relationship between the tissue anchors 46, 48 and the tissue engaging member 44 in the second position, desired electrode penetration depth and spacing are provided as well as desired myocardial tissue grip or capture. For example, shallow depth of electrode penetration may be desired in areas where the cardiac muscle is thin, such as the atria, or where pacing of the epicardium 28 is desired (near or over an endocardial scar). In contrast, greater depth of electrode penetration may be desired for a hypertrophic ventricle (abnormally thick ventricle) or where endocardial pacing is desired (near or under an endocardial scar, or to excite native purkinje conduction system.)

The spacing between the tissue anchors 46, 48 and the tissue engaging member 44 and between the tissue anchors 46, 48 themselves provides increased grip or capture of myocardial tissue 32 between the tissue anchors 46, 48 and the tissue engaging member 44. The amount of grip or capture may be increased or decreased by increasing or decreasing the spacing between the tissue anchors 46, 48, the spacing between the tissue anchors 46, 48 and the tissue engaging member 44, or the length of the tissue anchors 46, 48 and the tissue engaging member 44.

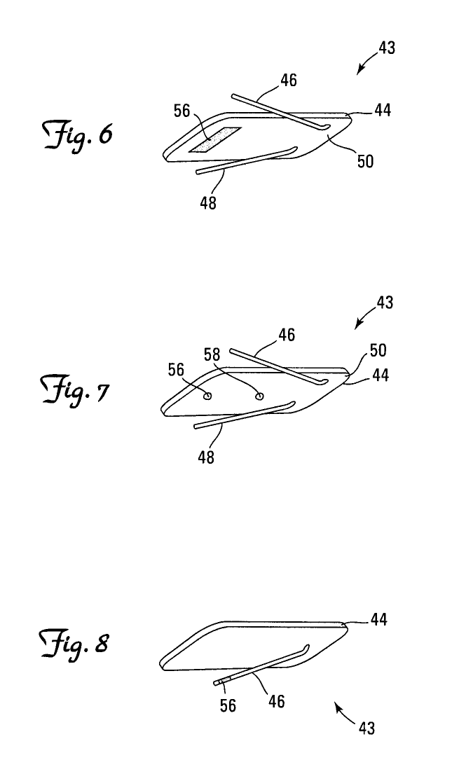

FIGS. 6 and 7 show other embodiments of the anchor mechanism 43, in which one or more electrodes 56, 58 are located on the tissue engaging surface 51 of the tissue engaging member 44. In these embodiments, the tissue anchors 46, 48 merely provide lead fixation rather than electrode sensing and pacing. The electrodes 56, 58 may be flat or coplanar with the tissue engaging surface 51, as illustrated with respect to FIG. 6, or may protrude from the tissue engaging surface 51 as is shown in FIG. 7.

FIG. 8 shows another embodiment of the anchor mechanism 43, in which a single tissue anchor 46 is provided. The tissue anchor 46 includes an electrode 56 as previously described. Alternately, or in addition, the tissue engaging member 44 may include an electrode on the tissue engaging surface (not shown).

Placement of the lead 36 of FIG. 1 may be accomplished by exposing a portion of the heart 12, for example by way of a sternotomy, thoracotomy or mini-thoracotomy. According to other embodiments, the heart 12 may be accessed via an endoscopic procedure according to known methods. Although shown implanted near the apex 38, the lead 36 may be implanted in the heart 12 anywhere pacing therapy is needed. Any known technique may be used to embed the anchors 46, 48 in the myocardium 32.

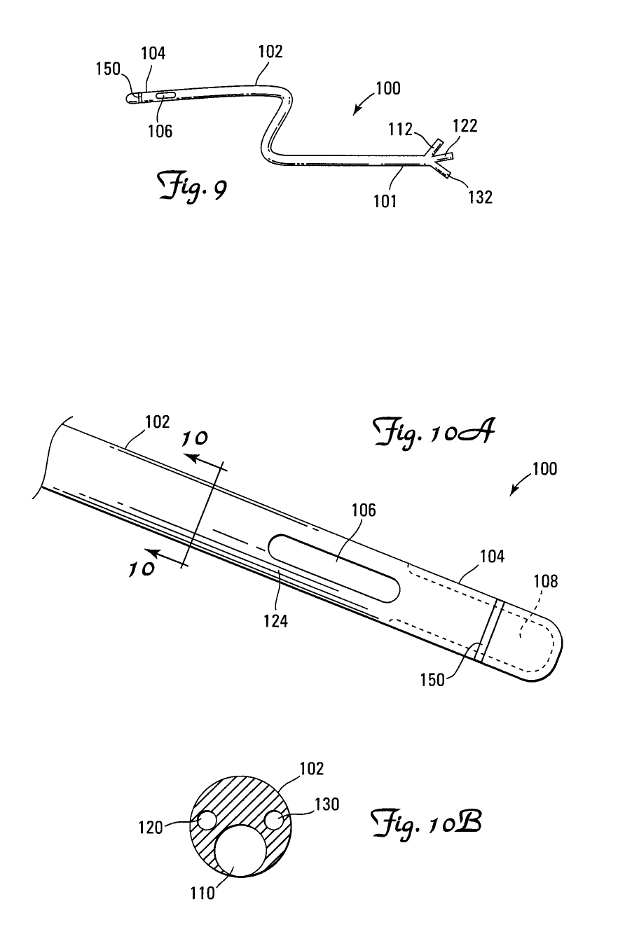

FIG. 9 shows an exemplary embodiment of a device 100 for inserting the lead 36 into the heart 12 to an operating position as shown in FIG. 1. As shown, the device 100 has an elongated device body 102 and extends from a proximal end 101 to a distal end 104. The device body 102 is sized so that the distal end 104 can be positioned at the surface of the heart 12 while the proximal end 101 is accessible from outside of the chest cavity. The device 100 has an opening 106 formed in the device body 102 near the distal end 104. In addition, a cavity 108 is formed in the device body 102 distal to the opening 106.

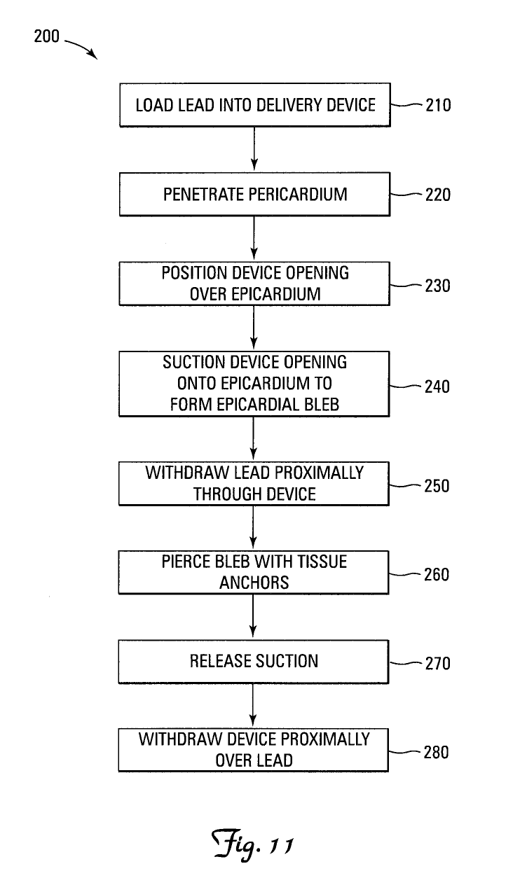

As shown in FIGS. 10A and 10B, the device 100 includes one or more lumens extending through the device body 102 from the proximal end to the distal end. Each lumen may provide access or delivery of payloads to the surface of the heart 12. In the illustrated embodiment, the device 100 includes four lumens. However, in other embodiments, the device 100 may include greater or few lumens depending upon the intended use of the device 100.

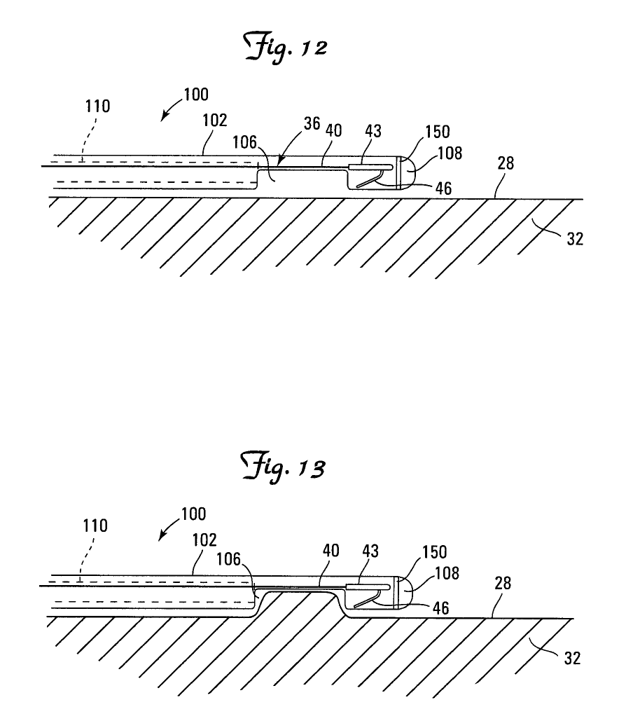

The device 100 includes a lead lumen 110 for delivering a lead, such as the lead shown in the preceding figures, to the heart 12. The lead lumen 110 extends from a proximal opening 112 to the device opening 106. As shown in FIG. 12, the lead 36 is inserted into the lead lumen 110 such that the anchor mechanism 43 is positioned within the cavity 108 distal to the device opening 106 and the lead extends proximally from the anchor mechanism 43 through the lead lumen 110. The tissue anchors 46, 48 (tissue anchor 48 not visible) are retained in a collapsed configuration while in the cavity 108 by the walls of the device body 102.

As further shown in FIGS. 10A and 10B, the device 100 further includes a vacuum lumen 120. The vacuum lumen 120 extends through the device body 102 from a proximal inlet 122 (see FIG. 9) adapted for coupling to a vacuum device to a distal outlet adjacent the device opening 106 (not shown). The vacuum lumen 120 is used to provide suction at the device opening 106. The suction is applied to the surface of the heart 12 to stabilize the distal end 104 of the delivery device 100 against the surface of the heart 12. The vacuum lumen 120 may also be adapted for evacuating or removing fluids from the heart 12.

In the illustrated embodiment, the device 100 further includes a visualization lumen 130. The visualization lumen 130 extends from a proximal port 132 (see FIG. 9) to a distal end (not shown) that is positioned adjacent the device opening 106 to allow a visualization device to view images of the heart 12 adjacent the device opening 106. The visualization device may be any such device known in the art, including, for example, an endoscope.

The device 100 as shown further includes an electrode 150 at or near the distal end 104 of the device body 102. The electrode 150 may be used for temporarily pacing the heart, or for mapping the electrical topography of the heart. In the illustrated embodiment, the electrode 150 is positioned distal to the device opening 106. In other embodiments, however, the electrode 150 may be positioned elsewhere on the device body 102. For example, the electrode 150 may be positioned adjacent to the device opening 106. In one embodiment, the device 100 further includes a needle or piercing instrument configured to form an access opening through the pericardium.

The lead 36 is inserted into the heart 12 with the device 100. The flowchart in FIG. 11 generally describes a method 200 of inserting the lead 36 into the heart 12 according to one embodiment of the invention. In particular, FIG. 11 describes a method of implanting the lead 36 so as to stimulate the epicardium 28 or myocardium 32 (depending upon the location of the electrode) of the heart 12. As shown in FIG. 12, the lead 36 is pre-loaded into the device 100 such that the anchor mechanism 43 is positioned within the cavity 108 and the conductors 40, 42 extend proximally from the anchor mechanism 43 through the lead lumen 110 (Block 210). The tissue anchors 46, 48 are retained in the compressed position within the cavity 108. With the lead 36 positioned in the lead lumen 110, the proximal end 101 of the device 100 is manipulated to maneuver the distal end 104 of the device 100 adjacent the pericardium of the heart 12.

An access opening in the pericardium of the heart 12 is formed (not shown) (Block 220). In one embodiment, the piercing structure of the device 100 is used to form an access opening in the pericardium. Alternately, a separate device may be employed to form an access opening in the pericardium. The proximal end 101 of the device 100 is manipulated to bring the distal end 104 of the device 100 through the pericardial access opening to the epicardial surface 28.

Introducers or other devices (not shown) may be employed to facilitate accessing the heart 12 and maneuvering the device 100 to the surface of the heart 12. Steering or other navigational devices such as guide wires, guide catheters, introducers or other devices as are known in the art (not shown) may be employed in conjunction with the device 100 to maneuver the distal end of the device 100 to the surface of the heart 12 (See FIG. 12). Published U.S. patent application Ser. No. 10/697,906, titled “Apparatus and Method for Endoscopic Cardiac Mapping and Lead Placement” filed Oct. 29, 2003, describes various structures and methods for placement of cardiac devices on a surface of the heart, and is hereby incorporated herein by reference in its entirety.

The electrode 150 can be brought into contact with the epicardium 28 to perform sensing and pacing functions prior to insertion of the lead 36. Additionally, acute therapeutic benefit at a particular site may be assessed using said embodiment. If acute benefit is unacceptable, the implant site may be changed prior to implanting the lead 36.

The device opening 106 is positioned over the epicardium 28 of the heart 12 and a vacuum or suction force is exerted on the epicardium 28 through the vacuum lumen 120 (see FIG. 13). The vacuum force draws the device opening 106 against the epicardium 28, stabilizing the device body 102 to the heart 12. As shown in FIG. 13, sufficient vacuum force is exerted to draw the epicardium 28 through the device opening 106 into the device body 102, forming an epicardial bleb 160 at the device opening 106 (Block 240). This stabilizes a portion of the epicardium 28 within the device body 102.

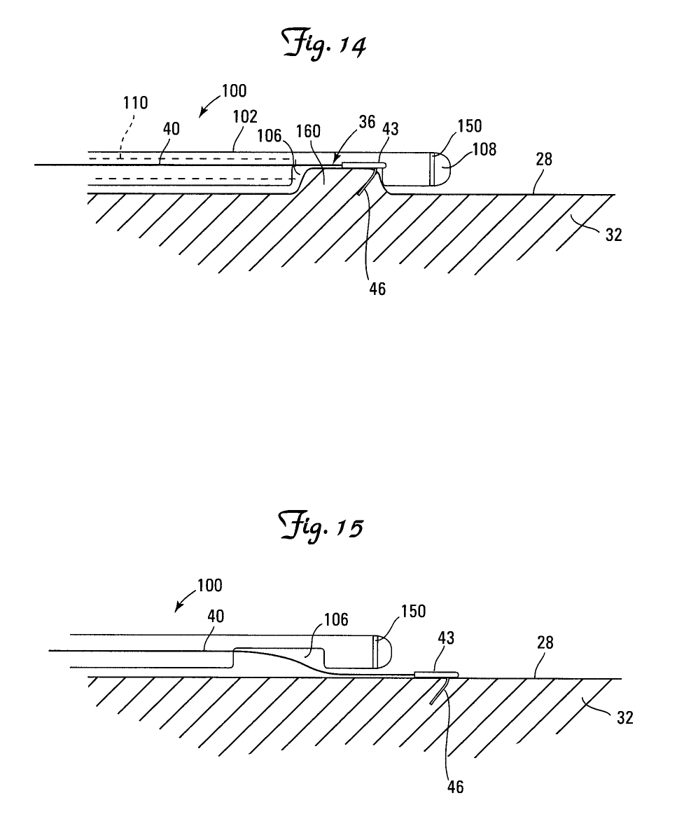

A proximal end 37 of the conductors 40, 42 (conductor 42 not visible) is tensioned to withdraw the lead 36 from the device 100 proximally (Block 250). This causes the anchor mechanism 43 to shift proximally within the cavity 108 and to pass over the device opening 106. As shown in FIG. 14, the tissue anchors 46, 48 are released from their compressed configuration at the device opening 106 and move outwardly under the biasing force previously described to deploy to the second position. The tissue anchors 46, 48 pierce the epicardial bleb 160 and penetrate the myocardium 32, thus snagging the lead 36 on the epicardium 28 of the heart 12 (Block 260). A portion of the epicardium 28 and myocardium 32 becomes wedged between the tissue anchor 46, 48 and the anchor mechanism 43, securing the distal end 39 of the lead 36 to the heart 12.

As shown in FIG. 15, once the tissue anchors 46, 48 pierce the epicardial bleb 160, the vacuum force is removed, releasing the bleb 160 from the device 100 (Block 270). The device 100 is then withdrawn proximally over the lead 36, which is fixed to the epicardium 28 of the heart 12 at the tissue anchors 46, 48 (Block 280). As the device 100 is withdrawn over the lead 36, a slight tension is exerted on the lead 36. This tension causes the tissue engaging member 44 to brace against the epicardium 28, increasing the fixation between the lead 36 and the heart 12. In one embodiment, the device 100 does not include a vacuum lumen and no bleb is formed. In this embodiment, the lead 36 is withdrawn proximally through the device 100 such that the tissue anchors 46, 48 snag on the epicardial surface of the heart 12 adjacent the opening 106 without the aid of bleb formation.

In other embodiments, the device 100 may be used to deploy the lead 36 onto the pericardial surface of the heart 12 (not shown). Thus, rather applying suction to the epicardium 28 so as to draw an epicardial bleb, suction is applied to the pericardium to draw a pericardial bleb. The lead 36 is deployed as previously described.

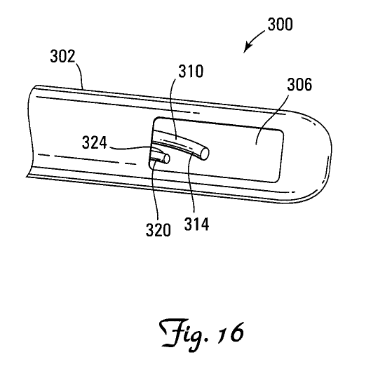

FIG. 16 shows a delivery device 300 according to another embodiment of the invention that is suited for implanting the lead 36 into the heart 12. The delivery device 300 is generally similar to the delivery device 100 shown in FIGS. 9 and 10A-10B. The delivery device 300 includes a distal device opening 306 and one or more lumens. In the embodiment shown, the device 300 includes a lead lumen 310 having a distal end 314 positioned over the device opening 306. The distal end 314 of the lead lumen 310 is angled towards the surface of the heart 12. The angle of the distal end 314 of the lead lumen 310 directs the lead 36 into the heart 12 such that the tissue anchors 46, 48 penetrate the heart 12 more easily (not shown). In the illustrated embodiment, the distal end 314 of the lead lumen 310 extends at an angle of about 45° relative to the device body 302. In other embodiments, however, the lead lumen 314 extends at an angle of from about 15° to about 60° relative to the device body 302. The device 300 further includes a vacuum lumen 320 having a distal end 324 positioned at the device opening 306.

The device 310 of FIG. 16 lacks the cavity 108 distal to the device opening 106 for housing the lead anchor mechanism 43 as is shown in the embodiment illustrated in FIG. 10A. The lead 36 is therefore positioned within the lead lumen 310 proximal to the device opening 306 as the delivery device 300 is maneuvered to the surface of the heart 12.

Similar to the method of lead delivery described with respect to FIG. 11, the delivery device 300 is maneuvered to the surface of the heart 12. The device opening 306 is positioned over the epicardium 28 of the heart 12 and a vacuum or suction force is exerted on the epicardium 28 through the vacuum lumen 320. The vacuum force draws the device opening 306 against the epicardium 28, stabilizing the device body 302 to the heart 12. Sufficient vacuum force may be exerted to draw the epicardium 28 through the device opening 306 into the device body 302, forming an epicardial bleb at the device opening 306.

Instead of being pulled proximally from the cavity 108 to the opening 106 so as to deploy the anchor mechanism 43, as is described with respect to FIG. 11, the lead 36 is advanced distally from the lead lumen 310 through the opening 306 towards the surface of the heart 12. The tissue anchors 46, 48 are released to pierce the epicardium 28 and to embed in the myocardium 32, thus securing the lead 36 to the heart 12. The lead 36 may be advanced distally and then slightly retracted proximally to facilitate piercing the epicardium 28. The delivery device 300 is then withdrawn. 10711 The delivery devices shown and described with respect to FIGS. 9-16 may be used in conjunction with the lead 36 shown in FIGS. 1-8. In addition, the delivery devices shown and described with respect to FIGS. 9-16 may be used to deliver other types of leads or payloads as are known in the art. Furthermore, the leads shown in FIGS. 1-8 may be implanted in the heart with other delivery devices as are known in the art.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An epicardial lead comprising:
an insulated conductor having a proximal end and a distal end;
an anchor assembly coupled to the distal end of the conductor, the anchor assembly including:
a tissue engaging member, and
a tissue anchor having a first end coupled to the tissue engaging member and a second end movable relative to the tissue engaging member, wherein the second end of the tissue anchor is biased away from the tissue engaging member to a position spaced apart from the tissue engaging member; and
an electrode positioned on the anchor assembly and in electrical communication with the conductor.

2. The epicardial lead of claim 1 wherein the electrode is on the tissue anchor.

3. The epicardial lead of claim 1 wherein the electrode is on a tissue engaging surface of the tissue engaging member.

4. The epicardial lead of claim 1 further comprising a second insulated conductor having a distal end coupled to the anchor assembly and a second electrode positioned on the anchor assembly and in electrical communication with the second conductor.

5. The epicardial lead of claim 1 wherein the anchor assembly further includes a second tissue anchor.

6. The epicardial lead of claim 5 wherein the second ends of the first and second tissue anchors are movable along a first arc away from the tissue engaging member and along a second arc away from one another.

7. The epicardial lead of claim 5 wherein the second ends of the first and second tissue anchors are spaced apart from one another by about 1 cm when the second ends of the tissue anchors are fully spaced apart from one another.

8. The epicardial lead of claim 5 wherein a first electrode is positioned on the first tissue anchor and a second electrode is positioned on the second tissue anchor.

9. The epicardial lead of claim 1 further comprising an anti-inflammatory coating on the tissue anchor.

10. A cardiac rhythm management system comprising:
a pulse generator for delivering therapy to a patient's heart;
an insulated conductor having a proximal end coupled to the pulse generator and a distal end adapted for implantation in the patient's heart;
an anchor assembly coupled to the distal end of the conductor, the anchor assembly including an anchor means coupled to a tissue engaging member; and
an electrode positioned on the anchor assembly and in electrical communication with the conductor.

11. The cardiac rhythm management system of claim 10 wherein the anchor means is biased away from the tissue engaging member.

12. The cardiac rhythm management system of claim 10 wherein the anchor means comprises a pair of anchors coupled to the tissue engaging member.

13. The cardiac rhythm management system of claim 10 wherein the electrode is positioned on the anchor means.

14. A method of implanting a lead into a space between a pericardium and an epicardium of a heart with a delivery device, the method comprising:
advancing a distal end of the delivery device into the space between the pericardium and the epicardium;
withdrawing the lead proximally relative to the delivery device such that a tissue anchor on a distal end of the lead is biased away from the lead into engagement with the epicardium; and
tensioning the lead such that the tissue anchor penetrates the myocardium and the epicardium is wedged between the tissue anchor and a distal end of the lead.

15. The method of claim 14 wherein advancing the distal end of the delivery device into the space between the pericardium and the epicardium further comprises using the delivery device to form a passageway through the pericardium.

16. The method of claim 15 wherein forming a passageway through the pericardium comprises:
suctioning a distal end of the delivery device to the pericardium;
drawing a bleb of the pericardium into a cavity at the distal end of the delivery device with the suction; and
piercing a passageway into the bleb with a needle.

17. The method of claim 14 further comprising:
suctioning a distal end of the delivery device to the epicardium;
drawing a bleb of the epicardium into a cavity at the distal end of the delivery device with the suction; and
withdrawing the lead proximally past the bleb such that the tissue anchor engages the bleb.

18. The method of claim 14 wherein withdrawing the lead proximally relative to the delivery device such that at least a first tissue anchor on a distal end of the lead is biased away from the lead into engagement with the epicardium further comprises positioning the tissue anchor over an opening in the delivery device to release the tissue anchor.

19. The method of claim 14 wherein the lead has first and second tissue anchors, wherein the method further comprises withdrawing the lead proximally relative to the delivery device such that the first and second tissue anchors are biased away from the lead and away from one another into engagement with the epicardium.

20. The method of claim 14 wherein tensioning the lead comprises withdrawing the delivery device proximally over the lead.
